(19) United States
(12) Patent Application Publication
(10) Pub. No.: US 2014/0275825 A1
(43) Pub. Date: Sep. 18, 2014

(54) METHODS AND SYSTEMS FOR LIGHT SIGNAL CONTROL IN A PHYSIOLOGICAL MONITOR

(71) Applicant: COVIDIEN LP, Mansfield, MA (US)

(72) Inventor: Daniel Lisogurski, Boulder, CO (US)

(73) Assignee: Covidien LP, Mansfield, MA (US)

(21) Appl. No.: 13/841,784

(22) Filed: Mar. 15, 2013

(51) Int. Cl. A61B 5/0205; A61B 5/1455

(52) U.S. Cl. CPC A61B 5/0205; A61B 5/14552
USPC 600/301; 600/323; 600/324

(57) ABSTRACT

A physiological monitoring system may select a light signal for determining a physiological parameter. In some embodiments, the monitoring system may select a received light signal for further processing based on a physiological metric such as blood oxygen saturation value, or based on a system metric such as a signal-to-noise ratio. In some embodiments, the system may determine a light drive parameter based on a received signal. For example, the system may select a received light signal for further processing in order to determine a physiological parameter.

The present disclosure relates to operating a physiological monitor, and more particularly relates to light signal control based on a metric in a pulse oximeter or other medical device.

### SUMMARY

Methods and systems are provided for light signal control in a physiological monitor.

In some embodiments, a monitor may receive two or more light signals attenuated by a subject, where each respective light signal corresponds to a different wavelength of light. A metric may be determined based on at least one of the two or more light signals, for example based on a blood oxygen saturation. At least one of the two or more light signals may be selected based on the metric, and a physiological parameter may be determined based on the selected signal. In an example, two received light signals may be used to determine a metric such as blood oxygen saturation, and the monitor may select a light signal with which to determine respiration rate based on the metric. In another example, a weighted combination of two or more received light signals may be determined, wherein the weights are based on the metric.

In some embodiments, a monitor may receive one or more light signals attenuated by a subject, where each respective light signal corresponds to a different wavelength of light. A metric may be determined based on at least one of the two or more light signals, for example based on a blood oxygen saturation value. A light drive parameter may be determined based on the metric. The light drive parameter may determine a wavelength of light to be emitted. A light drive signal may be generated based on the light drive parameter, and may be used to generate a photonic signal. A physiological parameter may be determined based on the photonic signal. In an example, the system may determine that a metric such as blood oxygen saturation is low. When blood oxygen saturation is low, the system may determine the light drive parameter such that a red wavelength of light is used to determine respiration rate.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 is a block diagram of an illustrative physiological monitoring system in accordance with some embodiments of the present disclosure;

FIG. 2A shows an illustrative plot of a light drive signal in accordance with some embodiments of the present disclosure;

FIG. 2B shows an illustrative plot of a detector signal that may be generated by a sensor in accordance with some embodiments of the present disclosure;

FIG. 3 is a perspective view of an embodiment of a physiological monitoring system in accordance with some embodiments of the present disclosure;

FIG. 4 shows an illustrative plot of signals in accordance with some embodiments of the present disclosure;

FIG. 5 shows an illustrative plot of a weighted signal combination in accordance with some embodiments of the present disclosure;

FIG. 6 shows an illustrative flow diagram including steps for selecting a light signal based on a metric in accordance with some embodiments of the present disclosure; and

FIG. 7 shows an illustrative flow diagram including steps for determining a light drive parameter based on a metric in accordance with some embodiments of the present disclosure.

### DETAILED DESCRIPTION OF THE FIGURES

The present disclosure is directed towards light signal control in a physiological monitoring system such as a medical device. A physiological monitoring system may monitor one or more physiological parameters of a patient, typically using one or more physiological sensors. For example, the physiological monitoring system may include a pulse oximeter. The system may include, for example, one or more light sources and a photosensitive detector.

In some embodiments, signals from a particular light source may be desired for use in determining certain physiological parameters. For example, blood oxygen saturation may be determined from both red and infrared (IR) light signals, while pulse rate, respiration rate, and blood pressure measurements may be determined from only a single light signal. In some embodiments, in order to determine a particular physiological parameter, the system may select one or more received light signals, may determine one or more light drive parameters, or any combination thereof.

In some embodiments, red or IR light signals may be selected based on the oxygen saturation levels of a patient's blood. Blood with a relatively high blood oxygen saturation may absorb IR light more strongly than red light. Thus, the IR light may be more sensitive than red light to pulsatile signals. For example, blood with a high blood oxygen saturation may have a relatively higher concentration of oxyhemoglobin and a relatively lower concentration of deoxyhemoglobin, resulting in higher IR sensitivity. Conversely, blood with relatively low blood oxygen saturation may absorb red light more strongly than infrared light. For example, blood with a low blood oxygen saturation may have a relatively lower concentration of oxyhemoglobin and a relatively higher concentration of deoxyhemoglobin, resulting in higher red sensitivity. Thus, it may be desirable to monitor activity with the more sensitive wavelength.

In some embodiments, the system may determine a metric used for selecting a light signal and/or for determining a light drive parameter. The metric may include a physiological parameter such as blood oxygen saturation, a monitor parameter such as power consumption, signal-to-noise ratio, signal quality, any other suitable parameter, or any combination thereof. For example, when the metric indicates a high blood oxygen saturation, information corresponding to an IR light signal may be selected to determine a particular physiological parameter, and when the metric indicates a low blood oxygen saturation, information corresponding to a red light signal may be selected to determine the parameter.

In some embodiments, the system may select two or more received light signals and may generate a weighted combination of the selected light signals in order to calculate a particular parameter. The weighting may be based on the metric. For example, when blood oxygen saturation levels are above a particular level (e.g., above 80%) the system may calculate a parameter such as continuous non-invasive blood pressure based on a 30%/70% combination of information corresponding to red and IR light signals, respectively. It will be understood that the weighting factors may range from 0% to 100% for each signal. Thus, the metric can be used to select and/or weight the light signals in order to, for example, improve the quality of the calculated parameter. The metric may additionally or alternatively be used to select and/or weight the light signals in order to reduce the power consumption of the system.

In some embodiments, the system may determine one or more light drive parameters based on the metric. The light drive parameters may, for example, determine a wavelength of light to be emitted. Light drive parameters may be used to generate light drive signals. The system may use the light drive signals to generate photonic signals with a light emitter such as an LED or laser. Light drive parameters may include a wavelength of light to be emitted, duty cycle, waveform shape, frequency of pulses, light drive schemes, other suitable light drive characteristics, or any combination thereof.

In some embodiments, the system may initially use two or more light sources to determine a metric, and may then use only a relatively smaller number of light sources (e.g., a single light source) based on the metric or other parameters. For example, the system may determine a blood oxygen saturation using a red and an IR light source, and may then monitor another physiological parameter such as respiration rate using only an IR light source with the red light switched off. In some embodiments, the system may not change the light drive signals but only alter the processing of received signals.

The foregoing techniques may be implemented in an oximeter. An oximeter is a medical device that may determine the oxygen saturation of an analyzed tissue. One common type of oximeter is a pulse oximeter, which may non-invasively measure the oxygen saturation of a patient's blood (as opposed to measuring oxygen saturation directly by analyzing a blood sample taken from the patient). Pulse oximeters may be included in patient monitoring systems that measure and display various blood flow characteristics including, but not limited to, the oxygen saturation of hemoglobin in arterial blood. Such patient monitoring systems may also measure and display additional physiological parameters, such as a patient's pulse rate, respiration rate, respiration effort, blood pressure, any other suitable physiological parameter, or any combination thereof. Exemplary embodiments of determining respiration rate are disclosed in Addison et al. U.S. Patent Publication No. 2011/0071406, published Mar. 24, 2011, which is hereby incorporated by reference herein in its entirety. Exemplary embodiments of determining respiration effort are disclosed in Addison et al. U.S. Patent Publication No. 2011/0004081, published Jan. 6, 2011, which is hereby incorporated by reference herein in its entirety. Exemplary embodiments of determining blood pressure are disclosed in Addison et al. U.S. Patent Publication No. 2011/0028854, published Feb. 3, 2011, which is hereby incorporated by reference herein in its entirety. Pulse oximetry may be implemented using a photoplethysmograph. Pulse oximeters and other photoplethysmograph devices may also be used to determine other physiological parameter and information as disclosed in: J. Allen, “Photoplethysmography and its application in clinical physiological measurement,” Physiol. Meas., vol. 28, pp. R1-R39, March 2007; W. B. Murray and P. A. Foster, “The peripheral pulse wave: information overlooked,” J. Clin. Monit., vol. 12, pp. 365-377, September 1996; and K. H. Shelley, “Photoplethysmography: beyond the calculation of arterial oxygen saturation and heart rate,” Anesth. Analg., vol. 105, pp. S31-S36, December 2007; all of which are incorporated by reference herein in their entireties.

An oximeter may include a light sensor that is placed at a site on a patient, typically a fingertip, toe, forehead or earlobe, or in the case of a neonate, across a foot or hand. The oximeter may use a light source to pass light through blood perfused tissue and photoelectrically sense the absorption of the light in the tissue. In addition, locations which are not typically understood to be optimal for pulse oximetry serve as suitable sensor locations for the blood pressure monitoring processes described herein, including any location on the body that has a strong pulsatile arterial flow. For example, additional suitable sensor locations include, without limitation, the neck to monitor carotid artery pulsatile flow, the wrist to monitor radial artery pulsatile flow, the inside of a patient's thigh to monitor femoral artery pulsatile flow, the ankle to monitor tibial artery pulsatile flow, and around or in front of the ear. Suitable sensors for these locations may include sensors for sensing absorbed light based on detecting reflected light. In all suitable locations, for example, the oximeter may measure the intensity of light that is received at the light sensor as a function of time. The oximeter may also include sensors at multiple locations. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate any of a number of physiological parameters, including an amount of a blood constituent (e.g., oxyhemoglobin) being measured as well as a pulse rate and when each individual pulse occurs.

In some embodiments, the photonic signal interacting with the tissue is of one or more wavelengths that are attenuated by the blood in an amount representative of the blood constituent concentration. Red and infrared (IR) wavelengths may be used because it has been observed that highly oxygenated blood will absorb relatively less red light and more IR light than blood with a lower oxygen saturation. By comparing the intensities of two wavelengths at different points in the pulse cycle, it is possible to estimate the blood oxygen saturation of hemoglobin in arterial blood.

The system may process data to determine physiological parameters using techniques well known in the art. For example, the system may determine blood oxygen saturation using two wavelengths of light and a ratio-of-ratios calculation. The system also may identify pulses and determine pulse amplitude, respiration, blood pressure, other suitable parameters, or any combination thereof, using any suitable calculation techniques. In some embodiments, the system may use information from external sources (e.g., tabulated data, secondary sensor devices) to determine physiological parameters.

In some embodiments, a light drive modulation may be used. For example, a first light source may be turned on for a first drive pulse, followed by an off period, followed by a second light source for a second drive pulse, followed by an off period. The first and second drive pulses may be used to determine physiological parameters. The off periods may be used to determine detected ambient signal levels, reduce overlap of the light drive pulses, allow time for light sources to stabilize, allow time for detected light signals to stabilize or settle, reduce heating effects, reduce power consumption, for any other suitable reason, or any combination thereof.

It will be understood that the light signal control techniques described herein are not limited to pulse oximeters and may be applied to any suitable medical and non-medical devices. For example, the system may include probes for parameters such as regional saturation (rSO2), respiration rate, respiration effort, continuous non-invasive blood pressure, oxygen saturation pattern detection, fluid responsiveness, cardiac output, any other suitable clinical parameter, or any combination thereof, and the system may control a light signal or combination of light signals in order to calculate such parameter(s).

The following description and accompanying FIGS. 1-7 provide additional details and features of some embodiments of the present disclosure.

FIG. 1 is a block diagram of an illustrative physiological monitoring system 100 in accordance with some embodiments of the present disclosure. System 100 may include a sensor 102 and a monitor 104 for generating and processing physiological signals of a subject. In some embodiments, sensor 102 and monitor 104 may be part of an oximeter. In some embodiments, all or some of sensor 102, monitor 104, or both, may be referred to collectively as processing equipment.

Sensor 102 of physiological monitoring system 100 may include light source 130 and detector 140. Light source 130 may be configured to emit photonic signals having one or more wavelengths of light (e.g. red and IR) into a subject's tissue. For example, light source 130 may include a red light emitting light source and an IR light emitting light source, e.g. red and IR light emitting diodes (LEDs), for emitting light into the tissue of a subject to generate physiological signals. In one embodiment, the red wavelength may be between about 600 nm and about 700 nm, and the IR wavelength may be between about 800 nm and about 1000 nm. It will be understood that light source 130 may include any number of light sources with any suitable characteristics. In embodiments where an array of sensors is used in place of single sensor 102, each sensor may be configured to emit a single wavelength. For example, a first sensor may emit only a red light while a second may emit only an IR light.

It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detector 140 may be chosen to be specifically sensitive to the chosen targeted energy spectrum of light source 130.

In some embodiments, detector 140 may be configured to detect the intensity of light at the red and IR wavelengths. In some embodiments, an array of sensors may be used and each sensor in the array may be configured to detect an intensity of a single wavelength. In operation, light may enter detector 140 after passing through the subject's tissue. Detector 140 may convert the intensity of the received light into an electrical signal. The light intensity may be directly related to the absorbance and/or reflectance of light in the tissue. That is, when more light at a certain wavelength is absorbed or reflected, less light of that wavelength is received from the tissue by detector 140. After converting the received light to an electrical signal, detector 140 may send the detection signal to monitor 104, where the detection signal may be processed and physiological parameters may be determined (e.g., based on the absorption of the red and IR wavelengths in the subject's tissue). In some embodiments, the detection signal may be preprocessed by sensor 102 before being transmitted to monitor 104.

In the embodiment shown, monitor 104 includes control circuitry 110, light drive circuitry 120, front end processing circuitry 150, back end processing circuitry 170, user interface 180, and communication interface 190. Monitor 104 may be communicatively coupled to sensor 102.

Control circuitry 110 may be coupled to light drive circuitry 120, front end processing circuitry 150, and back end processing circuitry 170, and may be configured to control the operation of these components. In some embodiments, control circuitry 110 may be configured to provide timing control signals to coordinate their operation. For example, light drive circuitry 120 may generate a light drive signal, which may be used to turn on and off the light source 130, based on the timing control signals. The front end processing circuitry 150 may use the timing control signals to operate synchronously with light drive circuitry 120. For example, front end processing circuitry 150 may synchronize the operation of an analog-to-digital converter and a demultiplexer with the light drive signal based on the timing control signals. In addition, the back end processing circuitry 170 may use the timing control signals to coordinate its operation with front end processing circuitry 150.

Light drive circuitry 120, as discussed above, may be configured to generate a light drive signal that is provided to light source 130 of sensor 102. The light drive signal may, for example, control the intensity of light source 130 and the timing of when light source 130 is turned on and off. When light source 130 is configured to emit two or more wavelengths of light, the light drive signal may be configured to control the operation of each wavelength of light. The light drive signal may comprise a single signal or may comprise multiple signals (e.g., one signal for each wavelength of light). An illustrative light drive signal is shown in FIG. 2A.

In some embodiments, control circuitry 110 and light drive circuitry 120 may generate light drive parameters based on a metric. For example, back end processing 170 may receive information about received light signals, determine light drive parameters based on that information, and send corresponding information to control circuitry 110.

FIG. 2A shows an illustrative plot of a light drive signal including red light drive pulse 202 and IR light drive pulse 204 in accordance with some embodiments of the present disclosure. Light drive pulses 202 and 204 are illustrated as square waves. As will be described in detail below, these pulses may include shaped waveforms rather than a square wave. The shape of the pulses may be generated by a digital signal generator, digital filters, analog filters, any other suitable equipment, or any combination thereof. For example, light drive pulses 202 and 204 may be generated by light drive circuitry 120 under the control of control circuitry 110. As used herein, drive pulses may refer to the high and low states of a shaped pulse, switching power or other components on and off, high and low output states, high and low values within a continuous modulation, other suitable relatively distinct states, or any combination thereof. The light drive signal may be provided to light source 130, including red light drive pulse 202 and IR light drive pulse 204 to drive red and IR light emitters, respectively, within light source 130. Red light drive pulse 202 may have a higher amplitude than IR light drive 204 since red LEDs may be less efficient than IR LEDs at converting electrical energy into light energy. In some embodiments, the output levels may be equal, may be adjusted for nonlinearity of emitters, may be modulated in any other suitable technique, or any combination thereof. Additionally, red light may be absorbed and scattered more than IR light when passing through perfused tissue.

When the red and IR light sources are driven in this manner they emit pulses of light at their respective wavelengths into the tissue of a subject in order generate physiological signals that physiological monitoring system 100 may process to calculate physiological parameters. It will be understood that the light drive amplitudes of FIG. 2A are merely exemplary and that any suitable amplitudes or combination of amplitudes may be used, and may be based on the light sources, the subject tissue, the determined physiological parameter, modulation techniques, power sources, any other suitable criteria, or any combination thereof.

The light drive signal of FIG. 2A may also include “off” periods 220 between the red and IR light drive pulse. “Off” periods 220 are periods during which no drive current may be applied to light source 130. “Off” periods 220 may be provided, for example, to prevent overlap of the emitted light, since light source 130 may require time to turn completely on and completely off. The period from time 216 to time 218 may be referred to as a drive cycle, which includes four segments: a red light drive pulse 202, followed by an “off” period 220, followed by an IR light drive pulse 204, and followed by an “off” period 220. After time 218, the drive cycle may be repeated (e.g., as long as a light drive signal is provided to light source 130). It will be understood that the starting point of the drive cycle is merely illustrative and that the drive cycle can start at any location within FIG. 2A, provided the cycle spans two drive pulses and two “off” periods. Thus, each red light drive pulse 202 and each IR light drive pulse 204 may be understood to be surrounded by two “off” periods 220. “Off” periods may also be referred to as dark periods, in that the emitters are dark or returning to dark during that period. It will be understood that the particular square pulses illustrated in FIG. 2A are merely exemplary and that any suitable light drive scheme is possible. For example, light drive schemes may include shaped pulses, sinusoidal modulations, time division multiplexing other than as shown, frequency division multiplexing, phase division multiplexing, any other suitable light drive scheme, or any combination thereof.

Referring back to FIG. 1, front end processing circuitry 150 may receive a detection signal from detector 140 and provide one or more processed signals to back end processing circuitry 170. The term “detection signal,” as used herein, may refer to any of the signals generated within front end processing circuitry 150 as it processes the output signal of detector 140. Front end processing circuitry 150 may perform various analog and digital processing of the detector signal. One suitable detector signal that may be received by front end processing circuitry 150 is shown in FIG. 2B.

FIG. 2B shows an illustrative plot of an detector current waveform 214 that may be generated by a sensor in accordance with some embodiments of the present disclosure. The peaks of detector current waveform 214 may represent current signals provided by a detector, such as detector 140 of FIG. 1, when light is being emitted from a light source. The amplitude of detector current waveform 214 may be proportional to the light incident upon the detector. The peaks of detector current waveform 214 may be synchronous with drive pulses driving one or more emitters of a light source, such as light source 130 of FIG. 1. For example, detector current peak 226 may be generated in response to a light source being driven by red light drive pulse 202 of FIG. 2A, and peak 230 may be generated in response to a light source being driven by IR light drive pulse 204. Valley 228 of detector current waveform 214 may be synchronous with periods of time during which no light is being emitted by the light source, or the light source is returning to dark, such as “off” period 220. While no light is being emitted by a light source during the valleys, detector current waveform 214 may not fall all of the way to zero.

It will be understood that detector current waveform 214 may be an at least partially idealized representation of a detector signal, assuming perfect light signal generation, transmission, and detection. It will be understood that an actual detector current will include amplitude fluctuations, frequency deviations, droop, overshoot, undershoot, rise time deviations, fall time deviations, other deviations from the ideal, or any combination thereof. It will be understood that the system may shape the drive pulses shown in FIG. 2A in order to make the detector current as similar as possible to idealized detector current waveform 214.

Referring back to FIG. 1, front end processing circuitry 150, which may receive a detection signal, such as detector current waveform 214, may include analog conditioning 152, analog-to-digital converter (ADC) 154, demultiplexer 156, digital conditioning 158, decimator/interpolator 160, and ambient subtractor 162.

Analog conditioning 152 may perform any suitable analog conditioning of the detector signal. The conditioning performed may include any type of filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof.

The conditioned analog signal may be processed by analog-to-digital converter 154, which may convert the conditioned analog signal into a digital signal. Analog-to-digital converter 154 may operate under the control of control circuitry 110. Analog-to-digital converter 154 may use timing control signals from control circuitry 110 to determine when to sample the analog signal. Analog-to-digital converter 154 may be any suitable type of analog-to-digital converter of sufficient resolution to enable a physiological monitor to accurately determine physiological parameters.

Demultiplexer 156 may operate on the analog or digital form of the detector signal to separate out different components of the signal. For example, detector current waveform 214 of FIG. 2B includes red component corresponding to peak 226, an IR component corresponding to peak 230, and at least one ambient component corresponding to valley 230. Demultiplexer 156 may operate on detector current waveform 214 of FIG. 2B to generate a red signal, an IR signal, a first ambient signal (e.g., corresponding to the ambient component corresponding to valley 230 that occurs immediately after the peak 226), and a second ambient signal (e.g., corresponding to the ambient component corresponding to valley 230 that occurs immediately after the IR component 230). Demultiplexer 156 may operate under the control of control circuitry 110. For example, demultiplexer 156 may use timing control signals from control circuitry 110 to identify and separate out the different components of the detector signal.

Digital conditioning 158 may perform any suitable digital conditioning of the detector signal. Digital conditioning 158 may include any type of digital filtering of the signal (e.g., low pass, high pass, band pass, notch, or any other suitable filtering), amplifying, performing an operation on the signal, performing any other suitable digital conditioning, or any combination thereof.

Decimator/interpolator 160 may decrease the number of samples in the digital detector signal. For example, decimator/interpolator 160 may decrease the number of samples by removing samples from the detector signal or replacing samples with a smaller number of samples. The decimation or interpolation operation may include or be followed by filtering to smooth the output signal.

Ambient subtractor 162 may operate on the digital signal. In some embodiments, ambient subtractor 162 may remove dark or ambient contributions to the received signal.

The components of front end processing circuitry 150 are merely illustrative and any suitable components and combinations of components may be used to perform the front end processing operations.

The front end processing circuitry 150 may be configured to take advantage of the full dynamic range of analog-to-digital converter 154. This may be achieved by applying gain to the detection signal by analog conditioning 152 to map the expected range of the detection signal to the full or close to full output range of analog-to-digital converter 154. The output value of analog-to-digital converter 154, as a function of the total analog gain applied to the detection signal, may be given as:

ADC Value=Total Analog Gain×[Ambient Light+LED Light]

Ideally, when ambient light is zero and when the light source is off, the analog-to-digital converter 154 will read just above the minimum input value. When the light source is on, the total analog gain may be set such that the output of analog-to-digital converter 154 may read close to the full scale of analog-to-digital converter 154 without saturating. This may allow the full dynamic range of analog-to-digital converter 154 to be used for representing the detection signal, thereby increasing the resolution of the converted signal. In some embodiments, the total analog gain may be reduced by a small amount so that small changes in the light level incident on the detector do not cause saturation of analog-to-digital converter 154.

However, if the contribution of ambient light is large relative to the contribution of light from a light source, the total analog gain applied to the detection current may need to be reduced to avoid saturating analog-to-digital converter 154. When the analog gain is reduced, the portion of the signal corresponding to the light source may map to a smaller number of analog-to-digital conversion bits. Thus, more ambient light noise in the input of analog-to-digital converter 154 may results in fewer bits of resolution for the portion of the signal from the light source. This may have a detrimental effect on the signal-to-noise ratio of the detection signal. Accordingly, passive or active filtering or signal modification techniques may be employed to reduce the effect of ambient light on the detection signal that is applied to analog-to-digital converter 154, and thereby reduce the contribution of the noise component to the converted digital signal.

Back end processing circuitry 170 may include processor 172 and memory 174. Processor 172 may be adapted to execute software, which may include an operating system and one or more applications, as part of performing the functions described herein. Processor 172 may receive and further physiological signals received from front end processing circuitry 150. For example, processor 172 may determine one or more physiological parameters based on the received physiological signals. Processor 172 may include an assembly of analog or digital electronic components. Processor 172 may calculate physiological information. For example, processor 172 may compute one or more of a pulse rate, respiration rate, blood pressure, or any other suitable physiological parameter. Processor 172 may perform any suitable signal processing of a signal, such as any suitable band-pass filtering, adaptive filtering, closed-loop filtering, any other suitable filtering, and/or any combination thereof. Processor 172 may also receive input signals from additional sources not shown. For example, processor 172 may receive an input signal containing information about treatments provided to the subject from User Interface 180. Additional input signals may be used by processor 172 in any of the calculations or operations it performs in accordance with back end processing circuitry 170 or monitor 104.

Memory 174 may include any suitable computer-readable media capable of storing information that can be interpreted by processor 172. In some embodiments, memory 174 may store calculated values, such as a pulse rate, a blood pressure, a blood oxygen saturation, a fiducial point location or characteristic, an initialization parameter, or any other calculated values, in a memory device for later retrieval. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system. Back end processing circuitry 170 may be communicatively coupled with use interface 180 and communication interface 190.

User interface 180 may include user input 182, display 184, and speaker 186. User interface 180 may include, for example, any suitable device such as one or more medical devices (e.g., a medical monitor that displays various physiological parameters, a medical alarm, or any other suitable medical device that either displays physiological parameters or uses the output of back end processing 170 as an input), one or more display devices (e.g., monitor, personal digital assistant (PDA), mobile phone, tablet computer, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

User input 182 may include any type of user input device such as a keyboard, a mouse, a touch screen, buttons, switches, a microphone, a joy stick, a touch pad, or any other suitable input device. The inputs received by user input 182 can include information about the subject, such as age, weight, height, diagnosis, medications, treatments, and so forth.

In an embodiment, the subject may be a medical patient and display 184 may exhibit a list of values which may generally apply to the patient, such as, for example, age ranges or medication families, which the user may select using user input 182. Additionally, display 184 may display, for example, an estimate of a subject's blood oxygen saturation generated by monitor 104 (referred to as an “SpO₂” measurement), pulse rate information, respiration rate information, blood pressure, any other parameters, and any combination thereof. Display 184 may include any type of display such as a cathode ray tube display, a flat panel display such a liquid crystal display or plasma display, or any other suitable display device. Speaker 186 within user interface 180 may provide an audible sound that may be used in various embodiments, such as for example, sounding an audible alarm in the event that a patient's physiological parameters are not within a predefined normal range.

Communication interface 190 may enable monitor 104 to exchange information with external devices. Communications interface 190 may include any suitable hardware, software, or both, which may allow monitor 104 to communicate with electronic circuitry, a device, a network, a server or other workstations, a display, or any combination thereof. Communications interface 190 may include one or more receivers, transmitters, transceivers, antennas, plug-in connectors, ports, communications buses, communications protocols, device identification protocols, any other suitable hardware or software, or any combination thereof. Communications interface 190 may be configured to allow wired communication (e.g., using USB, RS-232, Ethernet, or other standards), wireless communication (e.g., using WiFi, IR, WiMax, BLUETOOTH, UWB, or other standards), or both. For example, communications interface 190 may be configured using a universal serial bus (USB) protocol (e.g., USB 2.0, USB 3.0), and may be configured to couple to other devices (e.g., remote memory devices storing templates) using a four-pin USB standard Type-A connector (e.g., plug and/or socket) and cable. In some embodiments, communications interface 190 may include an internal bus such as, for example, one or more slots for insertion of expansion cards.

It will be understood that the components of physiological monitoring system 100 that are shown and described as separate components are shown and described as such for illustrative purposes only. In some embodiments the functionality of some of the components may be combined in a single component. For example, the functionality of front end processing circuitry 150 and back end processing circuitry 170 may be combined in a single processor system. Additionally, in some embodiments the functionality of some of the components of monitor 104 shown and described herein may be divided over multiple components. For example, some or all of the functionality of control circuitry 110 may be performed in front end processing circuitry 150, in back end processing circuitry 170, or both. In other embodiments, the functionality of one or more of the components may be performed in a different order or may not be required. In an embodiment, all of the components of physiological monitoring system 100 can be realized in processor circuitry.

FIG. 3 is a perspective view of an embodiment of a physiological monitoring system 310 in accordance with some embodiments of the present disclosure. In some embodiments, one or more components of physiological monitoring system 310 may include one or more components of physiological monitoring system 100 of FIG. 1. Physiological monitoring system 310 may include sensor unit 312 and monitor 314. In some embodiments, sensor unit 312 may be part of an oximeter. Sensor unit 312 may include one or more light source 316 for emitting light at one or more wavelengths into a subject's tissue. One or more detector 318 may also be provided in sensor unit 312 for detecting the light that is reflected by or has traveled through the subject's tissue. Any suitable configuration of light source 316 and detector 318 may be used. In an embodiment, sensor unit 312 may include multiple light sources and detectors, which may be spaced apart. Physiological monitoring system 310 may also include one or more additional sensor units (not shown) that may, for example, take the form of any of the embodiments described herein with reference to sensor unit 312. An additional sensor unit may be the same type of sensor unit as sensor unit 312, or a different sensor unit type than sensor unit 312 (e.g., a photoacoustic sensor). Multiple sensor units may be capable of being positioned at two different locations on a subject's body.

In some embodiments, sensor unit 312 may be connected to monitor 314 as shown. Sensor unit 312 may be powered by an internal power source, e.g., a battery (not shown). Sensor unit 312 may draw power from monitor 314. In another embodiment, the sensor may be wirelessly connected to monitor 314 (not shown). Monitor 314 may be configured to calculate physiological parameters based at least in part on data relating to light emission and acoustic detection received from one or more sensor units such as sensor unit 312. For example, monitor 314 may be configured to determine pulse rate, respiration rate, respiration effort, blood pressure, blood oxygen saturation (e.g., arterial, venous, or both), hemoglobin concentration (e.g., oxygenated, deoxygenated, and/or total), any other suitable physiological parameters, or any combination thereof. In some embodiments, calculations may be performed on the sensor units or an intermediate device and the result of the calculations may be passed to monitor 314. Further, monitor 314 may include display 320 configured to display the physiological parameters or other information about the system. In the embodiment shown, monitor 314 may also include a speaker 322 to provide an audible sound that may be used in various other embodiments, such as for example, sounding an audible alarm in the event that a subject's physiological parameters are not within a predefined normal range. In some embodiments, physiological monitoring system 310 may include a stand-alone monitor in communication with the monitor 314 via a cable or a wireless network link. In some embodiments, monitor 314 may be implemented as display 184 of FIG. 1.

In some embodiments, sensor unit 312 may be communicatively coupled to monitor 314 via a cable 324 at port 336. Cable 324 may include electronic conductors (e.g., wires for transmitting electronic signals from detector 318), optical fibers (e.g., multi-mode or single-mode fibers for transmitting emitted light from light source 316), any other suitable components, any suitable insulation or sheathing, or any combination thereof. In some embodiments, a wireless transmission device (not shown) or the like may be used instead of or in addition to cable 324. Monitor 314 may include a sensor interface configured to receive physiological signals from sensor unit 312, provide signals and power to sensor unit 312, or otherwise communicate with sensor unit 312. The sensor interface may include any suitable hardware, software, or both, which may be allow communication between monitor 314 and sensor unit 312.

In some embodiments, physiological monitoring system 310 may include calibration device 380. Calibration device 380, which may be powered by monitor 314, a battery, or by a conventional power source such as a wall outlet, may include any suitable calibration device. Calibration device 380 may be communicatively coupled to monitor 314 via communicative coupling 382, and/or may communicate wirelessly (not shown). In some embodiments, calibration device 380 is completely integrated within monitor 314. In some embodiments, calibration device 380 may include a manual input device (not shown) used by an operator to manually input reference signal measurements obtained from some other source (e.g., an external invasive or non-invasive physiological measurement system).

In the illustrated embodiment, physiological monitoring system 310 includes a multi-parameter physiological monitor 326. The monitor 326 may include a cathode ray tube display, a flat panel display (as shown) such as a liquid crystal display (LCD) or a plasma display, or may include any other type of monitor now known or later developed. Multi-parameter physiological monitor 326 may be configured to calculate physiological parameters and to provide a display 328 for information from monitor 314 and from other medical monitoring devices or systems (not shown). For example, multi-parameter physiological monitor 326 may be configured to display an estimate of a subject's blood oxygen saturation and hemoglobin concentration generated by monitor 314. Multi-parameter physiological monitor 326 may include a speaker 330.

Monitor 314 may be communicatively coupled to multi-parameter physiological monitor 326 via a cable 332 or 334 that is coupled to a sensor input port or a digital communications port, respectively and/or may communicate wirelessly (not shown). In addition, monitor 314 and/or multi-parameter physiological monitor 326 may be coupled to a network to enable the sharing of information with servers or other workstations (not shown). Monitor 314 may be powered by a battery (not shown) or by a conventional power source such as a wall outlet.

In some embodiments, all or some of monitor 314 and multi-parameter physiological monitor 326 may be referred to collectively as processing equipment.

In some embodiments, any of the processing components and/or circuits, or portions thereof, of FIGS. 1 and 3 may be referred to collectively as processing equipment. For example, processing equipment may be configured to amplify, filter, sample and digitize an input signal from sensor 102 (e.g., using an analog-to-digital converter), and calculate physiological information from the digitized signal. Processing equipment may be configured to generate light drive signals, amplify, filter, sample and digitize detector signals, and calculate physiological information from the digitized signal. In some embodiments, all or some of the components of the processing equipment may be referred to as a processing module.

FIG. 4 shows illustrative plot 400 of signals in accordance with some embodiments of the present disclosure. Plot 400 includes metric 402, red signal 404, and IR signal 406. It will be understood that while plot 400 shows red and IR light emission transitioning based on metric 402, the system may at times use both red and IR light sources to update the metric, while only one light source is used to calculate a physiological parameter such as heart rate. In some embodiments, metric 402 may be plotted as the metric amplitude versus time. In some embodiments, red signal 404 and IR signal 406 may be representative of an on/off condition for the signal versus time. For example, where the bar for red signal 404 is absent, the red light source may be switched off or the associated light signal may not be used for processing of physiological parameters. Similarly, where the bar for red signal 404 is present, the red light emitter may be on. For example, the red emitter may be switched on or off in a drive cycle that includes pulses from two or more emitters. It will be understood that these on and off states are merely exemplary and that the system may use any suitable states or gradations of states.

Metric 402 may include any suitable metric used for light signal control. The metric may include a physiological parameter such as blood gas saturation, blood oxygen saturation, respiration rate, respiration effort, pulse rate, blood velocity, blood pressure, ventilation parameter, exhaled gas analysis parameter, any other suitable physiological parameter, or any combination thereof. The metric may alternatively or additionally include a system parameter such as power consumption, available battery power, power source, signal quality, signal-to-noise ratio, percent modulation, received signal amplitude, received signal pulsatile amplitude, any other suitable parameter, or any combination thereof. In some embodiments, received signal pulsatile amplitude corresponds to the AC component of a received signal. It will be understood that the above parameters may be determined by the system, received from an external monitor, received from user input, determined by any other suitable technique, or any combination thereof. It will be understood that determining the metric may include any suitable processing such as averaging, filtering, combining of multiple signals, any other suitable technique, or any combination thereof.

In some embodiments, changes in metric 402 may be determined based on a comparison with a threshold, a slope, a change in slope, an absolute change, a trend, by any other suitable technique, or any combination thereof. As illustrated in plot 400, the system compares metric 402 to threshold 408. At time point 414, metric 402 crosses threshold 408. In some embodiments, a vector containing several metrics may be fed in to a pattern recognition type classifier or neural network which determines the appropriate wavelength or combination of wavelengths to use for at least one calculated parameter. In some embodiments, different physiological parameters may use different weightings, for example a first physiological parameter may be calculated based on pulsatile amplitude while a second physiological parameter may depend primarily on average signal amplitude (e.g., DC level). In an example, heart rate may be determined from the received light signal that has the largest pulsatile amplitude. In another example, the received light signal that has the largest shifts in baseline in the expected respiration frequency range (e.g., typically less than 20 breaths per minute) may be selected to determine respiration rate. In some embodiments, the system may determine a light drive parameter based on the metric in order to minimize the number of light sources used. The system may determine which light sources to use based in part on an estimate of the error in a determined physiological parameter if each respective light source is disabled. In some embodiments, the system may determine light drive wavelengths that consume relatively more power but provide more accurate parameters. In some embodiments, the system may prioritize power consumption over physiological parameter quality based on system design, user input, power source (e.g., battery or line voltage), any other suitable parameters, or any combination thereof.

In some embodiments, the system may include one or more threshold levels related to the metric. Reaching or crossing a threshold may result in a system setting being changed, for example, which light sources are turned on. Thresholds may be predetermined, set by the user, determined based on historical information, determined based on characteristics related to the patient, determined based on characteristics of the sensor and system, determined based on any other suitable criteria, or any combination thereof. Thresholds may be constant or vary in time. The threshold may include multiple threshold values corresponding to multiple characteristics. In some embodiments, the threshold may be adjusted or compensated based on system gain changes (e.g., a detector gain change).

In an example, metric 402 may be blood oxygen saturation and it may be compared to threshold 408. For example, threshold 408 may correspond to approximately 70-80% of maximum blood oxygen saturation. During time interval 410, when the blood oxygen saturation is greater than threshold 408, the system may operate in a first mode. For example, as illustrated, the system may use only an IR light signal to monitor a physiological parameter such as respiration rate. For example, the signal fluctuations in an IR signal at high blood oxygen saturation may be relatively larger than those of a red signal due to the increased absorbance of IR light by oxyhemoglobin as compared to red light absorption. At time point 414, the blood oxygen saturation may cross threshold 408. In some embodiments, this may be indicative of decreasing blood oxygen saturation. During time interval 412, the system may operate in a second mode. For example, as illustrated, the system may use only a red light signal to monitor respiration rate. For example, the signal fluctuations in a red signal may be relatively larger than those of an IR signal at low blood oxygen saturations due to the increased absorbance of red light by deoxyhemoglobin as compared to IR light absorption by deoxyhemoglobin.

Red signal 404 and IR signal 406 may include any suitable generation or processing of any suitable combination of light signals. As illustrated in FIG. 4, a change in selection of the red and IR signals is made based on a particular characteristic of metric 402, such as the level of metric 402. In an example, the signal used for monitoring a particular physiological characteristic may change at time point 414, where metric 402 crosses threshold 408. That is, before time point 414, IR signal 406 may be used to calculate a physiological parameter such as heart rate, and after time point 414, red signal 404 may be used to calculate the physiological parameter.

In some embodiments, the system may change a light drive parameter at time point 414. For example, the system may turn the IR light signal off and the red light signal on at time point 414. In another example, the system may vary the brightness, duty cycle, or other light drive parameters at time point 414. In another example described below, the change may be gradual rather than abrupt. It will be understood that red signal 404 and IR signal 406 may include any suitable wavelength, number, and type of light emitters and/or light signals. For example, the system may include three light signals, and the use of those signals may change at time point 414. It will also be understood that any suitable number of thresholds or other metric comparisons may be used to make any suitable number of determinations of a light drive parameter.

In some embodiments, selection of a light signal and/or determination of a light drive parameter may be based on any suitable combination of physiological parameters and system parameters. That is, metric 402 may represent a combination of physiological parameters (such as blood oxygen saturation, heart rate, blood pressure, age, sex, gender, or medical history) and system parameters (such as power consumption, power source, light source availability, probe or probes connected to the system). In some embodiments, the metric is based at least in part on a received light signal. In an example, a red wavelength LED may consume more power than an IR LED, and thus it may be desired to use the lowest power consuming technique possible. In some embodiments, an oxygen saturation value may be used as the metric, in order to select a more power efficient but less accurate wavelength light source (e.g., IR) to determine pulse rate when oxygen saturation is relatively high. In another example, respiration rate and battery level may be used together as a metric. For example, when the respiration rate is in a normal range, the system may determine to use a light source based on the battery level to conserve power if needed, but when the respiration rate is abnormal the system may give priority to physiological parameter quality in determining which light source to use. In another example, the system may determine to use a light source that provides the greatest pulsatile amplitude per unit of power consumed. In another example, the system may determine to use the light source that provides the greatest pulsatile amplitude.

FIG. 5 shows illustrative plot 500 of a weighted signal combination in accordance with some embodiments of the present disclosure. In some embodiments, selecting one or more light signals may include determining a weighted combination of two or more received signals. Weighting may be used to combine two or more signals rather than, for example, selecting one or the other. Plot 500 includes a metric 502, red signal 504, IR signal 506, weighting parameter 508, and combined signal 510. In some embodiments, plot 500 may illustrate the weighted combination of multiple light signals based on a metric. In some embodiments, plot 500 may illustrate that red signal 504 and IR signal 506 are emitting light throughout the time interval shown (e.g., in a drive cycle). In an example, when a signal quality metric associated with the received red light signal such as signal-to-noise increases, the weighting of the red light signal may be increased and the weighting of the IR light signal may be decreased. In another example, respiration rate may be determined based on a weighted combination of IR and red light, where the contribution of red relative to IR is increased as oxygen saturation decreases. In another example, red and IR light may be weighted and combined to determine respiration rate, where the weighting is adjusted to maximize the amplitude of baseline fluctuations. In another example, weights for combining red and IR light may be determined based on both power consumption and signal quality in order to maintain a required signal quality while minimizing power load. In another example, weights may be determined based on a power source such as battery or line voltage, such that power consumption may take priority over signal-to-noise when the power source is a battery.

Metric 502 may correspond to the information described for metric 402 of FIG. 4. Red signal 504 and IR signal 506 may correspond to red signal 404 of FIG. 4 and IR signal 406 of FIG. 4. In some embodiments, red signal 504 and IR signal 506 may be indicative of a received light signal, a light drive signal, any other suitable signal, or any combination thereof. The system may use weighting parameter 508 to generate a weighted combination of received light signals. For example, weighting parameter 508 may vary from 0 to 1 and may be used in Eq. 1:

C=x*(Red)+(1−x)(IR) (1)

where C is the combined signal, x is the weighting parameter, Red is the red signal, and IR is the IR signal. It will be understood that Eq. 1 is merely exemplary and that any suitable combination of any number of signals may be used, including more complex (e.g., non-linear) methods of combining these signals.

Weighting parameter 508 may be based on metric 502. For example, weighting parameter may be an inverted and smoothed version of metric 502. In another example, the weighting parameter may be changed based on threshold crossings as described above. In another example, the weighting parameter may be based on a mathematical equation including the metric, such as Eq. 1 above. It will be understood that the use of a single scalar as weighting parameter 508 is merely exemplary and that any suitable weighting parameter or parameters may be used.

Combined signal 510 illustrates the weighted combination of red signal 504 and IR signal 506 based on weighting parameter 508 using, for example, Eq. 1 above. For example, if the weighting parameter changes from x=0.1 to x=1, the contribution of red signal 504 to combined signal 510 changes from 10% to 100% across plot 500. It will be understood that the particular weighting parameter and the particular weighted combination illustrated in plot 500 is merely exemplary. In some embodiments, combined signal 510 may be used for further processing, for example, in determining a physiological parameter as described above. It will be understood that determining the weighted combination may be performed by any suitable circuitry illustrated in sensor 102 and monitor 104 of FIG. 1. For example, based on the value of the metric 502, a particular weighted combination of red, IR, and/or other signals may produce the best signal quality for determining a parameter such as respiration rate.

The flow diagrams 600 of FIGS. 6 and 700 of FIG. 7 describe embodiments of the present disclosure. It will be understood that the techniques of both flow diagrams may be used independently or in any suitable combination. It will also be understood that the particular steps of the flow diagrams are merely illustrative and that steps may be altered, added, removed, repeated, reordered, changed in any other suitable way, or any combination thereof.

FIG. 6 shows an illustrative flow diagram 600 including steps for selecting a light signal based on a metric in accordance with some embodiments of the present disclosure.

In step 602, the system receives one or more light signals. The one or more light signals may be received by, for example, detector 140 of FIG. 1. In some embodiments, the received light signals may correspond to an emitted photonic signal from, for example, light source 130 of FIG. 1. In some embodiments, the received light signals may be processed using processing equipment such as that described in front end processing 150 of FIG. 1, back end processing equipment 170 of FIG. 1, any other suitable processing equipment, or any combination thereof. For example, the detector current waveform 214 of FIG. 2 may be generated by a photoelectric sensor. This waveform may be filtered, demultiplexed, and/or otherwise suitable processed to determine one or more light signals corresponding to, for example, particular wavelength of light. In some embodiments, received light signals may be processed to remove drive pulse modulation as illustrated in FIG. 2A. In some embodiments, the system may receive a red light signal, an IR light signal, any other suitable light signal, or any combination thereof.

In step 604, the system determines a metric based at least in part on at least one light signal. The metric may include physiological parameters and/or system parameters as described above. The metric may include any suitable metric as described in relation to metric 402 of FIG. 4. In some embodiments, the metric may be determined based on the light signals received in step 602. For example, the system may receive a red and IR light signal in step 602, and may use a blood oxygen saturation value determined from the received red and IR light signals as the metric. As described above, the system may combine multiple parameters to determine the metric. In some embodiments, metrics may include system parameters such as a power source, power level, power consumption, or other system parameters.

In step 606, the system selects at least one light signal based on the metric. For example, in FIG. 4, the system selected red light signal 404 or IR light signal 406. The signal or signals may be selected for further processing. In some embodiments, the system may compare the metric to a trend, target value, threshold, or other suitable characteristic, and may select one or more light signals based on the comparison. For example, where the metric is blood oxygen saturation, the system may select an IR light signal when the blood oxygen saturation is above 80%, and may select a red light signal when the blood oxygen saturation is below 80%. The selected signal is used for further processing to determine a physiological parameter (e.g., pulse rate, respiration rate, respiration effort, blood pressure, any other suitable parameter, and any suitable combination thereof). In another example, the metric may include power consumption, and the system may determine that the best signal quality at a particular power consumption may be attained by using a particular light source or combination of light sources. The system may then select such a light source based on the metric.

In some embodiments, selecting a light signal may include determining a weighted combination of more than one light signal. A weighting parameter may be determined as described for weighting parameter 508 of FIG. 5. For example, a weighting parameter may be determined based on the metric. In some embodiments, the weighting parameter may be used to determine a weighted average of two or more signals using Eq. 1. It will be understood that in using three or more signals, the system may use multiple weighting parameters and an equation different than that of Eq. 1. For example, three light sources may be combined based on multiple weighting parameters, such that:

$$C = {x}_{1} * ( Signal 1 ) + {x}_{2} * ( Signal 2 ) + … + {x}_{n} * ( Signal n ) \\ given \text{:} {\sum }_{i = 1}^{n} {x}_{i} = 1 \qquad ( 2 )$$

where C is the combined signal, xₙ is a weighting parameter, Signal, is a signal being combined, and n is the number of signals being combined. Non-linear combinations are also possible by one skilled in the art with the benefit of this disclosure.

In some embodiments, multiple wavelengths of light emitters may be available, and less that the total number of emitters may be used. For example, three wavelengths of emitters may be available, and two of the three emitters may be selected. Selection may be based on oxygen saturation levels, noise, signal confidence, system design, physiological parameters being determined, any other suitable parameter, or any combination thereof. In an example, a sensor such as sensor 102 of FIG. 1 may include a red emitter, a near IR emitter, and a far IR emitter. The system may select the red emitter signal and near IR emitter signal, and not use the far IR emitter signal, based on any suitable metric, such as blood oxygen saturation level or other patient conditions.

In step 608, the system determines a physiological parameter based on the selected light signal. Physiological parameters may include blood gas saturation, blood pressure, pulse rate, respiratory rate, respiratory effort, any other suitable physiological parameter, or any combination thereof. Parameters may include any suitable parameter described in relation to back end processing 170 of FIG. 1, and monitor 314 and/or monitor 326 of FIG. 3.

In an example of the foregoing steps of flow diagram 600, the system may be used to determine respiration rate based on the metric of blood oxygen saturation. In this example, in step 602, the system receives a red light signal and an IR light signal. In step 604 the system determines blood oxygen saturation based on the received light signals. In step 606, the system uses the metric to determine the weighted combination of red and IR signals that provides the best quality respiration rate. For example, at 85% blood oxygen saturation, the weighted combination may be 25% red and 75% IR. In step 608, the system determines the respiration rate using the weighted combination.

In another example of the foregoing steps of flow diagram 600, the metric may be the pulsatile amplitude of the received light signals. The system may, in step 606, select a received light signal with the greatest pulsatile amplitude, and determine a physiological parameter in step 608 using that selected signal.

It will be understood that in some embodiments, the system may initially use two or more light signals in determining a metric, and then use only one light emitter to generate only one light signal, thus reducing power consumption. The system may continue to determine the metric using only one light signal, may intermittently turn on the other light signal to periodically determine the metric with both signals, may determine the metric by any other suitable technique, or any combination thereof.

FIG. 7 shows an illustrative flow diagram 700 including steps for determining a light drive parameter based on a metric in accordance with some embodiments of the present disclosure.

In step 702, the system receives one or more light signals. The signals may be attenuated by a subject. The one or more light signals may each correspond to a wavelength of light. In some embodiments, the system may receive a light signal as described for one or more of the light signals in step 602 of FIG. 6.

In step 704, the system determines a metric based on the at least one light signal received in step 702. The metric may include physiological parameters and/or system parameters as described above. The metric may include any suitable metric as described in relation to metric 402 of FIG. 4. In some embodiments, the metric may be determined based on the one or more light signals received in step 702. For example, the system may receive a red and IR light signal in step 702, and may use a blood oxygen saturation value determined from the received red and IR light signals as the metric. As described above, the system may combine multiple parameters to determine the metric. In some embodiments, metrics may include system parameters such as a power source, power level, power consumption, or other system parameters.

In step 706, the system determines a light drive parameter based on the metric determined in step 704. In some embodiments, a light drive parameter may determine one or more wavelengths of light to be emitted (e.g., which emitters to be used). For example, the system may determine, based on the metric, that only the red light is needed, and may turn off all other emitters to save power, improve signal quality, for any other suitable reason, or any combination thereof. It will be understood that turning off a light source is merely exemplary and that the system may implement other variations to the light drive parameters. In some embodiments, where for example two light sources are used to determine the metric but only one light source is selected to determine a parameter, the system may periodically turn on both light sources to determine the metric, while in the intervening times using only the selected one light source to determine the parameter. In some embodiments, the light drive parameter may be determined by back end processing circuitry 170 of FIG. 1 and implemented by control circuitry 110 of FIG. 1 and light drive circuitry 120 of FIG. 1.

In some embodiments, the system may turn a particular wavelength of light emitter on or off, or use a weighted combination of wavelengths, based on the metric. In some embodiments, emitters with emission wavelengths that are not required or desired for the current oxygen saturation level or other metric may be switched off and/or replaced by more relevant, sensitive, useful, or otherwise desired wavelengths. For example, at high oxygen saturation levels, red and far IR wavelength emitters may be desirable for determining oxygen saturation, while at lower oxygen saturation levels, near IR and far IR wavelength emitters may be desirable. In another example, the particular wavelengths used may be determined based on the physiological parameter or parameters determined in step 708, as described below.

In some embodiments, the system determines a light drive parameter based in part on what emitter wavelengths are available in a sensor. For example, the system may include information regarding a connected sensor such as what wavelength emitters are present, the emission efficiency of the emitters, the number of emitters, the voltage and current requirements of the emitters, any other suitable characteristics, or any combination thereof. In some embodiments, sensor parameters may be encoded in a sensor, such that the system receives sensor parameter information from the sensor. In some embodiments, the system receives user input regarding the connected sensor, for example, the system may receive a model number and/or particular emitter parameters. In some embodiments, determining a light drive parameter may be based on a physiological parameter such as oxygen saturation and emitter parameters associated with the sensor. For example, LED voltage requirements and LED efficiency may be used with the current oxygen saturation level in selecting an emitter. In some embodiments, determining light drive parameters may be based on encoded information including a predetermined switching point and/or a formula for determining a switching point. For example, a sensor may include encoded information to switch from a first wavelength emitter to a second wavelength emitter if oxygen saturation falls below 80%.

In an example of determining a light drive parameter, the system may, based on the metric, turn on only a red light emitter. In another example, the metric may be oxygen saturation, and the system may turn off an IR emitter and turn on a red emitter based on the oxygen saturation value. In another example, the metric may be a signal-to-noise ratio, and the system may change from a 680 nm red emitter to a 650 nm red emitted based on the signal-to-noise ratio. In another example, the metric may include battery level, and the system may switch to a higher efficiency light source based on the metric. It will be understood that the aforementioned are merely exemplary and that the system may determine any suitable light drive parameter or parameters based on any suitable metric or metrics.

In step 708, the system emits light using the light drive parameter determined in step 706. In some embodiments, the system may generate at least one light drive signal based on the light drive parameter determined in step 706. The light drive signal may be used to generate at least one photonic signal, which may be attenuated by the subject. For example, the light drive signal may drive an emitter such as light source 130 of FIG. 1 to emit a photonic signal. The photonic signal may interact with a test subject and be partially attenuated. The system may detect the attenuated signal using, for example, a photodetector. In some embodiments, the system may determine a physiological parameter based on the photonic signal. Physiological parameters may include, for example, oxygen saturation, blood pressure, heart rate, respiratory rate, and respiratory effort. Physiological parameters may include any suitable parameter described in relation to back end processing 170 of FIG. 1, and monitor 314 and/or monitor 326 of FIG. 3.

The foregoing is merely illustrative of the principles of this disclosure and various modifications may be made by those skilled in the art without departing from the scope of this disclosure. The above described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

What is claimed:

1. A method of determining a physiological parameter of a subject, the method comprising:
receiving, using processing equipment, two or more light signals attenuated by a subject, wherein each respective light signal corresponds to a different wavelength of light;
determining, using processing equipment, a metric based at least in part on at least one of the two or more received light signals;
selecting, using processing equipment, at least one of the two or more light signals based on the metric; and
determining, using the processing equipment, a physiological parameter of the subject based on the at least one selected light signal.

2. The method of claim 1, wherein determining the metric comprises determining a metric selected from the group consisting of oxygen saturation level, pulsatile amplitude of at least one light signal, signal quality, percent modulation, and any combination thereof.

3. The method of claim 1, wherein the metric is further based at least in part on a system parameter.

4. The method of claim 1, further comprising determining a weighting factor based at least in part on the metric, wherein selecting the at least one light signal comprises determining a weighted combination of two or more light signals based on the weighting factor.

5. The method of claim 1, wherein determining a physiological parameter comprises determining a physiological parameter selected from the group consisting of oxygen saturation, blood pressure, heart rate, respiratory rate, respiratory effort, and any combination thereof.

6. A system for determining a physiological parameter of a subject, the system comprising:
processing equipment configured to:
receive two or more light signals attenuated by a subject, wherein each respective light signal corresponds to a different wavelength of light;
determine a metric based at least in part on at least one of the two or more received light signals;
select at least one of the two or more light signals based on the metric; and
determine a physiological parameter of the subject based on the at least one selected light signal.

7. The system of claim 6, wherein the metric is selected from the group consisting of oxygen saturation level, pulsatile amplitude of at least one light signal, signal quality, percent modulation, and any combination thereof.

8. The system of claim 6, wherein the metric is determined further based at least in part on a system parameter.

9. The system of claim 6, wherein the processing equipment is further configured to determine a weighting factor based at least in part on the metric, and wherein the processing equipment is configured to select the at least one light signal by determining a weighted combination of two or more light signals based on the weighting factor.

10. The system of claim 6, wherein the physiological parameter is selected from the group consisting of oxygen saturation, blood pressure, heart rate, respiratory rate, respiratory effort, and any combination thereof.

11. A method of determining a physiological parameter of a subject, the method comprising:
receiving, using processing equipment, one or more light signals attenuated by a subject;
determining, using processing equipment, a metric based on at least one of the one or more light signals;
determining, using processing equipment, a light drive parameter based on the metric, wherein the light drive parameter determines a wavelength of light to be emitted;
generating, using processing equipment, at least one light drive signal based on the light drive parameter, wherein the at least one light drive signal is used to generate at least one photonic signal attenuated by a subject, wherein the at least one photonic signal is of at least the wavelength of light to be emitted; and
determining, using the processing equipment, a physiological parameter of the subject based at least in part on the at least one photonic signal.

12. The method of claim 11, further comprising generating the at least one photonic signal from at least one light emitter.

13. The method of claim 11, wherein determining the metric comprises determining a metric selected from the group consisting of oxygen saturation, pulsatile amplitude of at least one light signal, signal quality, and any combination thereof.

14. The method of claim 11, wherein the metric is further based at least in part on a system parameter.

15. The method of claim 11, wherein determining a physiological parameter comprises determining a physiological parameter selected from the group consisting of oxygen saturation, blood pressure, heart rate, respiratory rate, respiratory effort, and any combination thereof.

16. A system for determining a physiological parameter of a subject, the system comprising:
processing equipment configured to:
receive one or more light signals attenuated by a subject;
determine a metric based on at least one of the one or more light signals;
determine a light drive parameter based on the metric, wherein the light drive parameter determines a wavelength of light to be emitted;
generate at least one light drive signal based on the light drive parameter, wherein the at least one light drive signal is used to generate at least one photonic signal attenuated by a subject, wherein the at least one photonic signal is of at least the wavelength of light to be emitted; and
determine a physiological parameter of the subject based at least in part on the at least one photonic signal.

17. The system of claim 16, wherein the processing equipment is further configured to generate the at least one photonic signal from at least one light emitter.

18. The system of claim 16, the metric is selected from the group consisting of oxygen saturation, pulsatile amplitude of at least one light signal, signal quality, and any combination thereof.

19. The method of claim 16, wherein the metric is further based at least in part on a system parameter.

20. The method of claim 16, wherein the physiological parameter is selected from the group consisting of oxygen saturation, blood pressure, heart rate, respiratory rate, respiratory effort, and any combination thereof.
